(19) United States
(12) Patent Application Publication
(10) Pub. No.: US 2026/0048017 A1
(43) Pub. Date: Feb. 19, 2026

(54) SYSTEM AND METHOD FOR DRY COATING OF SUBSTRATE MATERIALS

(71) Applicant: Yarmouk University, Irbid (JO)

(72) Inventors: Derar Moh'd Ibrahim OMARI, Irbid (JO); Sami Mustafa Ali QASSIM, Irbid (JO); Hussein Taleb Ahmad ALTALAFHA, Irbid (JO)

(73) Assignee: Yarmouk University, Irbid (JO)

(21) Appl. No.: 18/808,387

(22) Filed: Aug. 19, 2024

(51) Int. Cl. A61K 9/20; A61K 9/28; A61K 31/167

(52) U.S. Cl. CPC A61K 9/2086; A61K 9/28; A61K 31/167

(57) ABSTRACT

A system for dry coating pharmaceutical solid dosage forms, including a fluidized bed coating unit, a plasticizer delivery system, a coating material reservoir, and an air compressor with advanced pneumatic controls. The present disclosure also provides a method for dry coating pharmaceutical solid dosage forms using the system of the present disclosure. The system and method of the present disclosure ensure precise material delivery and uniform coating application, significantly reducing processing time and improving coating efficiency compared to conventional solvent-based coating methods.

### TECHNICAL FIELD

The present disclosure relates to pharmaceutical coating technologies, and more particularly to a system and a method for dry coating pharmaceutical solid dosage forms using a fluidized bed, aimed at enhancing coating efficiency and uniformity while reducing processing time compared to conventional solvent-based coating systems and methods.

### BACKGROUND

Dry coating is a practical method that applies coatings to particles without using liquid solvents. This method ensures precise delivery of powders and plasticizers in a controlled environment, allowing for consistent, multilayer coatings at lower temperatures. By avoiding solvents, dry coating helps prevent common problems such as particle agglomeration and equipment adhesion. This makes dry coating an excellent choice for pharmaceutical industries.

The Polish patent publication number PL215692 discloses a system for dry coating processes for fine particles with micro and nano powders. It features a spout-fluidized bed with an air management setup that includes pneumatic distribution system, an air dryer, multiple filters, and controlled temperature air for coating processes. The system offers multilayer coating option at low temperature, without solvent involvement, and can be used for coating fine particles.

A study by Sakae Obara et al. in 1999 discloses an innovative dry coating method for enteric coatings that utilizes a cellulose derivative. The method directly applies a coating polymer powder to pharmaceutical cores, such as beads and tablets, using equipment like centrifugal granulator, fluidized bed, or tablet-coating machine. While it requires a higher amount of coating material for effective gastric resistance, the technique cuts down processing time and may be practical for water-sensitive active ingredients.

None of the prior art documents discloses an apparatus for dry coating of diverse solid pharmaceutical dosage forms with different types of coating materials using a fluidized bed that integrates advanced components for precise material delivery and pneumatic control, suitable for a wide range of pharmaceutical substrates including granules, tablets, mini tablets, capsules, pellets, small particles, and powders.

### SUMMARY

Therefore, it is an object of the present disclosure to provide a system and a method for dry coating of pharmaceutical solid dosage forms that is designed to improve the efficiency and uniformity of the coating processes without the use of liquid solvents.

In aspects of the present disclosure, there is provided a system for dry coating of substrate materials, the system may include a fluidized bed coating unit having an air handling system, a coating powder pneumatic feeder, and a concentric air and plasticizer nozzle feeder; a coating material reservoir having a first chamber configured to contain a coating material, a second chamber configured to contain an anti-adherent material, and an air regulator; a plasticizer delivery unit having a plasticizer container configured to contain a plasticizer material, and a peristaltic pump configured to deliver the plasticizer material to the fluidized bed coating unit through the concentric air and plasticizer nozzle feeder; and an air compressor configured to provide the fluidized bed coating unit and the coating material reservoir with air, and to deliver the plasticizer material to the fluidized bed coating unit by providing air through the concentric air and plasticizer nozzle feeder.

In aspects of the present disclosure, the air compressor may be in operable connection with the fluidized bed coating unit and/or the coating material reservoir.

In aspects of the present disclosure, the coating material reservoir may be in operable connection with the fluidized bed coating unit.

In aspects of the present disclosure, the plasticizer delivery unit may be in operable connection with the fluidized bed coating unit.

In aspect of the present disclosure, the air handling system may include an air inlet, filters configured to provide clean air to the fluidized bed coating unit; and a heating unit configured to control the temperature of the air supplied to the fluidized bed coating unit.

In some aspects, the first chamber of the coating material reservoir may have an upper portion having an air inlet and a lower portion having a coating material outlet in operable connection with the coating powder pneumatic feeder.

In some aspects, the second chamber of the coating material reservoir may have an upper portion having an air inlet and a lower portion having an anti-adherent material outlet in operable connection with the coating powder pneumatic feeder.

In yet other aspects of the present disclosure, the air compressor may further include a first outlet valve configured to control air flow to the air feeder, and a second outlet valve configured to control air flow to the air regulator.

In aspects of the present disclosure, the air regulator may be configured to selectively control air pressure delivered from the air compressor to either the first chamber or the second chamber based on a set of pre-identified operational requirements.

In some aspects, the air regulator is connected via a pipe that can be selectively attached to either the air inlet of the first chamber or the air inlet of the second chamber of the coating material reservoir.

In some aspects of the present disclosure, the air compressor may provide air to the air regulator when connected to the air inlet of the upper portion of the first chamber to carry the coating powder throughout the coating material outlet of the lower portion of the first chamber to the fluidized bed coating unit via the coating powder pneumatic feeder.

In some aspects, the air compressor may provide air through the pipe connected to the air regulator when connected to the air inlet of the upper portion of the second chamber to carry the anti-adherent material throughout the coating material outlet of the lower portion of the second chamber to the fluidized bed coating unit via the coating powder pneumatic feeder.

In some aspects, the coating powder may be selected from a group including polymeric powders, sugar powders, excipient powders, enteric coating powders, effervescent powders, colorant powders, gastro-resistance powders, combinations thereof, or any other suitable material.

In some aspects, the anti-adherent material may be selected from a group including magnesium stearate, talc, silicon dioxide, stearic acid, cornstarch, calcium silicate, sodium stearyl fumarate, or combinations thereof, or any other suitable material.

In some aspects, the plasticizer material may be selected from a group including glycerin, sorbitol, polyethylene glycol, glycerol triacetate, propylene glycol, polypropylene glycol, ethylene glycol, butyl phthalyl butyl glycolate, vegetable oils, castor oil, phthalates, triethyl citrate, or combinations thereof, or any other suitable material.

In some aspects, the substrate material may be a sloid pharmaceutical dosage form.

In some aspects, the substrate material may be selected from a group including granules, tablets, mini tablets, capsules, pellets, small particles and powders.

Other aspects of the present disclosure provide a method for dry coating pharmaceutical solid dosage forms using the system described herein, the method may include the steps of:
Placing a substrate material within the fluidized bed coating unit;
Initiating fluidizing air through the air inlet of the air handling system, passing though the filters to prepare the substrate material for coating;
Initiating heating via the heating unit in the air handling system;
Loading the plasticizer material into the plasticizer container that is operatively connected to the peristaltic pump;
Loading the coating powder into the first chamber and an anti-adherent material into the second chamber of the coating material reservoir;
Activating the peristaltic pump and the air compressor to spray the plasticizer material onto the substrate material through the concentric air and plasticizer nozzle feeder; and
Operating the air compressor to regulate airflow through the air regulator, thereby transporting the coating powder from the first chamber or the anti-adherent material from the second chamber, as required, to the fluidized bed coating unit via the coating powder pneumatic feeder, where it is applied to the substrate material.

### BRIEF DESCRIPTION OF THE DRAWINGS

The disclosure will now be described with reference to the accompanying drawings, without however limiting the scope of the disclosure thereof, and in which:

FIG. 1 illustrates a schematic block diagram of a system for dry coating of pharmaceutical solid dosage forms configured in accordance with one or more embodiments of the present disclosure.

FIG. 2 illustrates another schematic diagram of the system for dry coating of pharmaceutical solid dosage forms, including its components, a fluidized bed coating unit, a coating material reservoir unit, an air compressor, and a plasticizer delivery unit.

FIG. 3 illustrates a schematic diagram of a coating material reservoir of a system for dry coating of pharmaceutical solid dosage forms, the coating material reservoir being configured in accordance with one or more embodiments of the present disclosure.

FIG. 4. illustrates another schematic diagram showing the internal configuration of the coating material reservoir of FIG. 3 detailing a first chamber and a second chamber configured in accordance with one or more embodiments of the present disclosure.

FIG. 5 illustrates a schematic diagram of a plasticizer delivery unit of a system for dry coating of pharmaceutical solid dosage forms, the plasticizer delivery unit being configured in accordance with one or more embodiments of the present disclosure.

FIG. 6 illustrates a schematic diagram of a fluidized bed coating unit of a system for dry coating of pharmaceutical solid dosage forms, the fluidized bed coating unit being configured in accordance with one or more embodiments of the present disclosure.

FIG. 7 illustrates a flowchart of a method for dry coating of pharmaceutical dosage forms using a system for dry coating of pharmaceutical solid dosage forms, the system being configured in accordance with one or more embodiments of the present disclosure.

### DETAILED DESCRIPTION

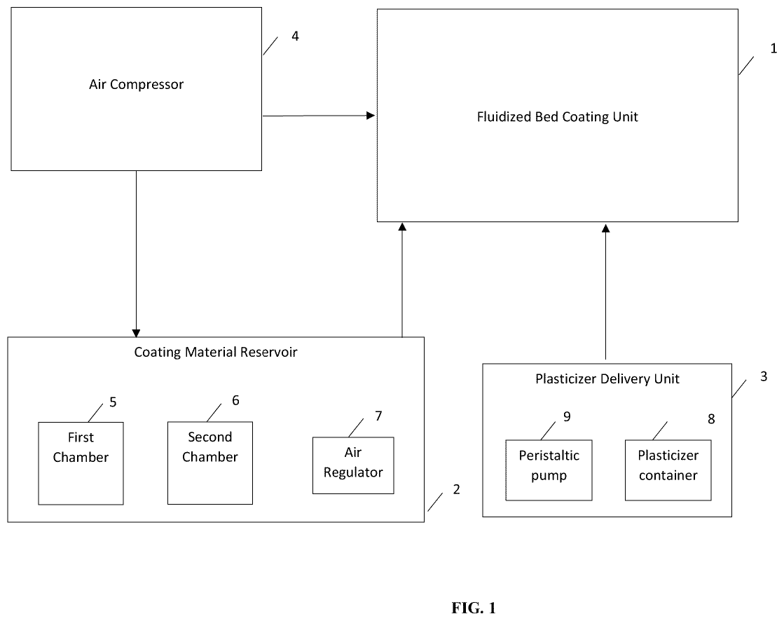

FIGS. 1-6 illustrate a system for dry coating of pharmaceutical solid dosage forms configured in accordance with embodiments of the present disclosure. The system includes a fluidized bed coating unit 1, a coating material reservoir 2, a plasticizer delivery unit 3, and an air compressor 4. The system in embodiments of the present disclosure is designed to improve the efficiency and uniformity of traditional coating processes without the use of liquid solvents, in which the fluidized bed coating unit 1, the coating material reservoir 2, the plasticizer delivery unit 3, and the air compressor 4 may be operably connected to each other in series or in parallel.

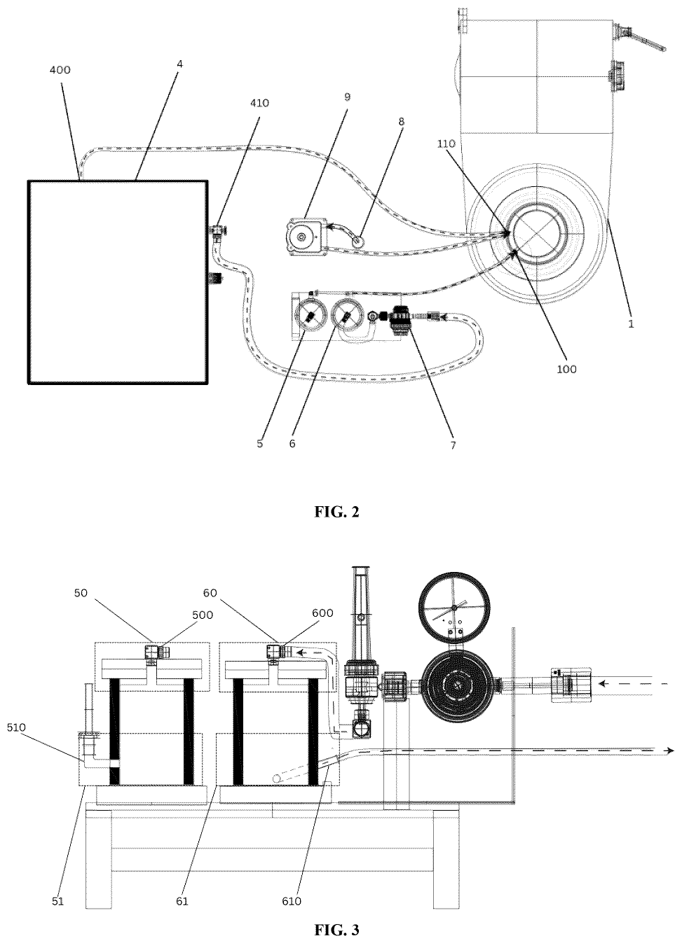

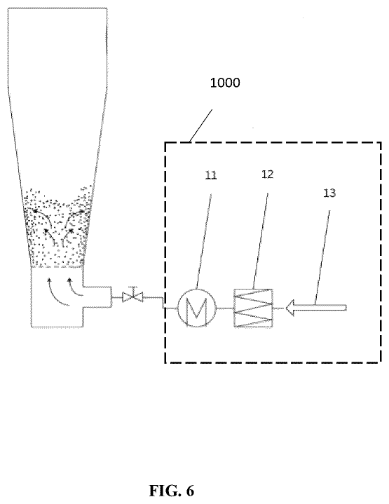

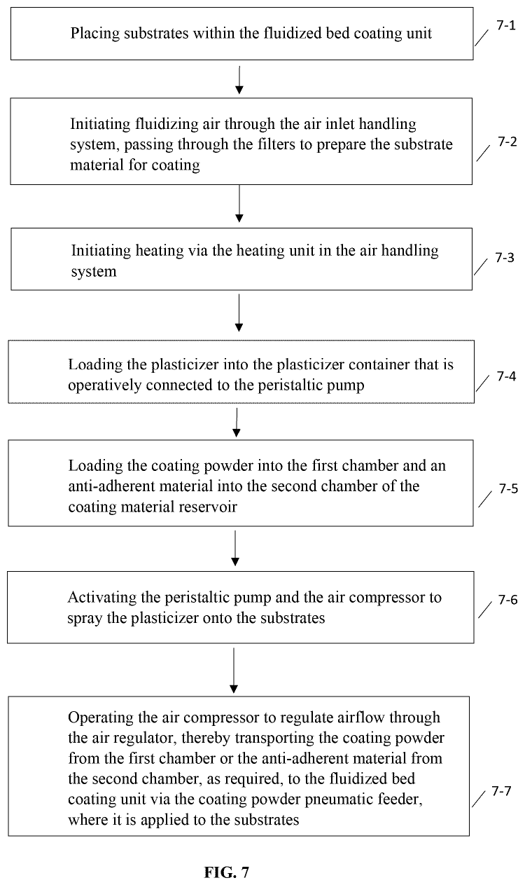

Reference is now being made to FIGS. 2 and 6 with continued reference to FIG. 1. In embodiments of the disclosure, the fluidized bed coating unit 1 may include an air handling system 1000 that may include an air inlet 13, filters 12, and a heating unit 11. In order to maintain substrates in a fluidized state, the fluidized bed coating unit 1 may further include a coating powder pneumatic feeder 100 configured to deliver coating materials from the coating material reservoir 2 to the fluidized bed coating unit 1 and to ensure precise deposition of the coating materials onto substrate materials. Additionally, the fluidized bed coating unit 1 may further include a concentric air and plasticizer nozzle feeder 110 configured to deliver plasticizers from the plasticizer delivery unit 3, wherein these plasticizers are required to enhance the adhesion of the coating materials onto the substrates.

Reference is now being made to FIG. 2. In embodiments of the disclosure, the air compressor 4 may include a first outlet valve 400 configured to supply air to the fluidized bed coating unit 1 through the concentric air and plasticizer nozzle feeder 110 and a second outlet valve 410 configured to supply air to the coating material reservoir 2.

Reference is now being made to FIGS. 1, 3-4. In embodiments of the disclosure, the coating material reservoir 2 may include a first chamber 5 configured to enclose coating materials, and a second chamber 6 configured to enclose anti-adherent materials. The first chamber 5 has an upper portion 50 with an air inlet 500 and a lower portion 51 with a coating material outlet 510, and the second chamber has an upper portion 60 with an air inlet 600 and a lower portion 61 with an anti-adherent material outlet 610.

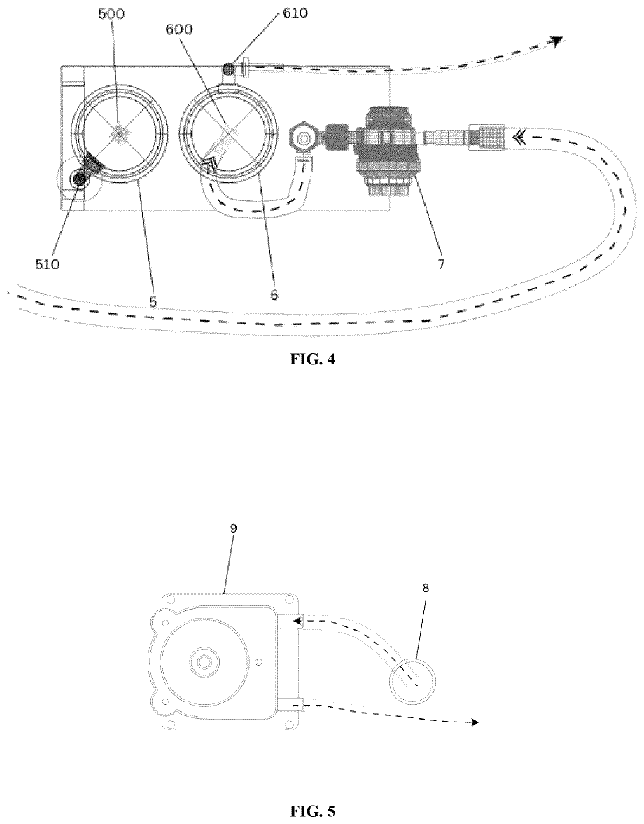

Reference is now being made to FIG. 4 with continued reference to FIG. 1. In embodiments of the disclosure, the coating material reservoir 2 may further include an air regulator 7 configured to selectively control air delivered from the air compressor 4 to the first or second chambers 5, 6 of the coating material reservoir 2 based on predefined operational requirements.

In embodiments of the disclosure, the air regulator 7 is selectively connected to the air inlet 500 of the first chamber 5 or the air inlet 600 of the second chamber 6.

Reference is now being made to FIGS. 2, 4. In embodiments of the disclosure, the air compressor 4 may be configured to provide air through the air regulator 7 when connected to the air inlet 500 of the first chamber 5 to carry the coating materials throughout the coating material outlet 510 of the first chamber 5 to the fluidized bed coating unit 1 via the coating powder pneumatic feeder 100.

In embodiments of the disclosure, the air compressor 4 may be configured to provide air through the air regulator 7 when connected to the air inlet 600 of the second chamber 6 to carry the anti-adherent materials throughout the anti-adherent material outlet 610 of the second chamber 6 to the fluidized bed coating unit 1 via the coating powder pneumatic feeder 100.

Reference is now being made to FIG. 5 with continued reference to FIG. 1. In embodiments of the disclosure, the plasticizer delivery unit 3 may include a plasticizer container 8 and a peristaltic pump 9. The plasticizer delivery unit 3 may be configured to deliver plasticizers from the plasticizer container 8 through the peristaltic pump 9 to the fluidized bed coating unit 1.

In some embodiments of the disclosure, the system has adjustable parameters that may include air flow rates, temperature settings, and material delivery speeds. Such adjustable parameters can be finely tuned to accommodate specific coating requirements of various pharmaceutical forms.

In some embodiments of the disclosure, the coating powder may be selected from a group including polymeric powders, sugar powders, excipient powders, thermal plastics, enteric coating powders, effervescent powders, colorant powders, gastro-resistance powders, combinations thereof, or any other suitable coating powder.

In some embodiments of the disclosure, the anti-adherent material may be selected from a group including magnesium stearate, talc, silicon dioxide, stearic acid, cornstarch, calcium silicate, sodium stearyl fumarate, combinations thereof, or any other suitable anti-adherent material.

In some embodiments of the disclosure, the plasticizer may be selected from a group including glycerin, sorbitol, polyethylene glycol, glycerol triacetate, propylene glycol, polypropylene glycol, ethylene glycol, butyl phthalyl butyl glycolate, vegetable oils, castor oil, phthalates, triethyl citrate, combinations thereof, or any other suitable plasticizer.

Reference is now being made to FIG. 7, which illustrates a flowchart of a method for dry coating pharmaceutical solid dosage forms using the system described above, the method may include the steps of: Placing a substrate material within the fluidized bed coating unit (process block 7-1); initiating fluidizing air through the air inlet of the air handling system, passing through the filters to prepare the substrate material for coating (process block 7-2); heating, using the heating unit, in the air handling system (process block 7-3); loading the plasticizer into the plasticizer container that is operatively connected to the peristaltic pump (process block 7-4); loading the coating powder into the first chamber and an anti-adherent material into the second chamber of the coating material reservoir (process block 7-5); activating the peristaltic pump and the air compressor to spray the plasticizer onto the substrate material (process block 7-6); and operating the air compressor to regulate airflow through the air regulator, thereby transporting the coating powder from the first chamber or the anti-adherent material from the second chamber, as required, to the fluidized bed coating unit via the coating powder pneumatic feeder, where it is applied to the substrates (process block 7-7).

In some embodiments of the disclosure, the substrates may be selected from a group including granules, tablets, mini tablets, capsules, pellets, small particles and powders.

### Example 1

### Comparison of Dry Coating and Conventional Coating Using Tablets

In this example, a comparative study was conducted between conventional aqueous dispersion coating and the system and method of the present disclosure for dry coating of Paracetamol tablets (6 mm, 150 mg wt). For the conventional coating process, Eudragit RS aqueous dispersion and Glycerin as a plasticizer were used, with results showing a significant amount of time required for the coating process, as illustrated in Table 1. On the other hand, Eudragit RS coating powder (60 micron) and Glycerin as a plasticizer were used in the system and method of the present disclosure. Results show that a substantial reduction in coating time while achieving higher weight gains, as illustrated in Table 2. The Coat/Core Ratio (“CCR”) was measured to indicate the ratio of the weight of the coating material to the weight of the core. This significant decrease in coating time underscores the efficiency of the disclosed apparatus.

TABLE 1
Conventional Coating Results

| | Tablet (Batch) | % wt gained | Time taken | |
|---|---|---|---|---|
| Batch No. | wt (g) | (CCR) | (min) | Date |
| 1C | 316 | 1.5 | 40 | 4 Apr. 2022 |
| 2C | 250 | 4.6 | 90 | 5 Apr. 2022 |
| 3C | 250 | 8.1 | 100 | 6 Apr. 2022 |

TABLE 2
Dry Coating Results

| | Tablet (Batch) | % wt gained | Time taken | |
|---|---|---|---|---|
| Batch No. | wt (g) | (CCR) | (min) | Date |
| 1D | 300 | 7.8 | 10 | 28 Mar. 2022 |
| 2D | 250 | 7.6 | 19 | 7 Apr. 2022 |
| 3D | 250 | 11.6 | 13 | 12 Apr. 2022 |

### Example 2

### Drug Layering and Multi-Layering on Pellets

In this example, pellets were coated using Paracetamol powder to evaluate the layering capability of the system and method of the present disclosure. Both single and multiple layering were studied. In the single layering process, Paracetamol powder was sieved through a 60-micron sieve and layered on pellet surfaces using the same mechanism as in dry coating. Results showed significant weight gains in relatively short times, as illustrated in Table 3. For multi-layering, additional layers were applied to the previously coated pellets, demonstrating the system's capability to handle multiple layers effectively, as illustrated in Table 4. This indicates that the pellets demonstrated a large surface area for receiving multiple layers, indicating the versatility of the system and method of the present disclosure in handling different dosage forms and coating complexities.

TABLE 3
Single Layer Coating

| | pellets wt | | Time taken | |
|---|---|---|---|---|
| Batch No | (g) | % wt gained | (min) | Date |
| 1SL | 217 | 24.8 | 20 | 7 Mar. 2021 |
| 2SL | 300 | 9.9 | 14 | 30 Mar. 2021 |
| 3SL | 300 | 18.3 | 20 | 27 Apr. 2021 |

TABLE 4
Multiple Layers Coating for Batch No. 1SL

| | Pellets | | % wt | Cumulative wt % | Time taken | |
|---|---|---|---|---|---|---|
| Trial | wt (g) | Plasticizer | gained | gained | (min) | Date |
| 1^{st} Layer | 217.0 | Glycerin | 24.8 | 24.8 | 25.5 | 7 Mar. 2021 |
| 2^{nd} Layer | 243.7 | Glycerin | 25.6 | 56.7 | 21.0 | 8 Mar. 2021 |
| 3^{rd} Layer | 299.7 | Glycerin | 12.6 | 72.3 | 21.5 | 9 Mar. 2021 |
| 4^{th} Layer | 336.0 | Glycerin | 08.3 | 83.0 | 12.0 | 25 Apr. 2021 |
| 5^{th} Layer | 348.0 | Glycerin | 11.0 | 103.0 | 20.5 | 26 Apr. 2021 |

### Example 3

### Dry Coating of Larger Tablets

In this example, the effect of tablet size on coating efficiency using the system and method of the present disclosure was evaluated by coating larger Paracetamol tablets (12 mm diameter, 450 mg average wt). The materials used included Eudragit RL as the coating polymer, Talc as an anti-adherent, and Triethyl Citrate (“TEC”) as a plasticizer. Results (Table 5) indicated that larger tablets required longer coating times due to their lower surface area, suggesting the need for process adjustments based on tablet size.

TABLE 5
Coating of lager tables

| | Tablet (batch) | % wt gain | Time taken | |
|---|---|---|---|---|
| Batch No. | wt (g) | (CCR) | (min) | Date |
| 1DL | 300 | 6.2 | 25 | 30 May 2021 |
| 2DL | 300 | 8.3 | 39 | 1 Jun. 2021 |
| 3DL | 300 | 3.3 | 20 | 2 Jun. 2021 |

While embodiments of the present disclosure have been described in detail and with reference to specific embodiments thereof, it will be apparent to one skilled in the art that various additions, omissions, and modifications can be made without departing from the spirit and scope thereof.

The singular forms “a,” “an,” and “the” include plural referents unless the context clearly dictates otherwise.

As used herein, the term “and” in the claims is used to mean “and/or” unless explicitly indicated to refer to collective nature only.

What is claimed is:

1. A system for dry coating of substrate materials, the system comprises a fluidized bed coating unit having an air handling system;
a coating powder pneumatic feeder;
a concentric air and plasticizer nozzle feeder;
a coating material reservoir having a first chamber with an upper portion and a lower portion, the first chamber is configured to enclose a coating material, a second chamber with an upper portion and a lower portion, the second chamber is configured to contain an anti-adherent material, and an air regulator;
a plasticizer delivery unit having a plasticizer container configured to contain a plasticizer material, and a peristaltic pump configured to deliver the plasticizer material to the fluidized bed coating unit through the concentric air and plasticizer nozzle feeder; and
an air compressor configured to provide the coating material reservoir with air, and to help to deliver the plasticizer material to the fluidized bed coating unit by providing air through the concentric air and plasticizer nozzle feeder.

2. The system of claim 1, wherein the air compressor is in operable connection with the fluidized bed coating unit and the coating material reservoir.

3. The system of claim 1, wherein the coating material reservoir is in operable connection with the fluidized bed coating unit.

4. The system of claim 1, wherein the plasticizer delivery unit is in operable connection with the fluidized bed coating unit.

5. The system of claim 1, wherein the air handling system comprises an air inlet, filters configured to provide clean air to the fluidized bed coating unit, and a heating unit configured to control temperature of air supplied to the fluidized bed coating unit.

6. The system of claim 1, wherein the upper portion of the first chamber of the coating material reservoir has an air inlet, and wherein the lower portion of the first chamber has a coating material outlet in operable connection with the coating powder pneumatic feeder.

7. The system of claim 1, wherein the upper portion of the second chamber of the coating material reservoir has an air inlet, and wherein the lower portion of the second chamber has an anti-adherent material outlet in operable connection with the coating powder pneumatic feeder.

8. The system of claim 1, wherein the air compressor comprises a first outlet valve configured to control air flow to the concentric air and plasticizer nozzle feeder, and a second outlet valve configured to control air flow to the air regulator.

9. The system of claim 1, wherein the air regulator is configured to selectively control air pressure delivered from the air compressor to either the first chamber or the second chamber based on a set of pre-identified operational requirements.

10. The system of claim 1, wherein the air regulator is connected via a pipe that can be selectively attached to either an air inlet of the first chamber or an air inlet of the second chamber of the coating material reservoir.

11. The system of claim 1, wherein the air compressor provides air to the air regulator when connected to an air inlet of the upper portion of the first chamber to carry the coating powder throughout a coating material outlet of the lower portion of the first chamber to the fluidized bed coating unit via the coating powder pneumatic feeder.

12. The system of claim 1, wherein the air compressor provides air through the pipe connected to the air regulator when connected to an air inlet of the upper portion of the second chamber to carry the anti-adherent material throughout a coating material outlet of the lower portion of the second chamber to the fluidized bed coating unit via the coating powder pneumatic feeder.

13. The system of claim 1, wherein the coating powder is selected from a group comprising polymeric powders, sugar powders, excipient powders, thermal plastics, enteric coating powders, effervescent powders, colorant powders, gastro-resistance powders, or combinations thereof.

14. The system of claim 1, wherein the anti-adherent material is selected from a group comprising magnesium stearate, talc, silicon dioxide, stearic acid, cornstarch, calcium silicate, sodium stearyl fumarate, or combinations thereof.

15. The system of claim 1, wherein the plasticizer is selected from a group comprising glycerin, sorbitol, polyethylene glycol, glycerol triacetate, propylene glycol, polypropylene glycol, ethylene glycol, butyl phthalyl butyl glycolate, vegetable oils, castor oil, phthalates triethyl citrate, or combinations thereof.

16. The system of claim 1, wherein the substrate material is a solid pharmaceutical dosage form.

17. The system of claim 1, wherein the substrate material is selected from a group comprising granules, tablets, mini tablets, capsules, pellets, small particles and powders.

18. A method for dry coating pharmaceutical solid dosage forms using the system of claim 1, the method comprises the steps of:
Placing a substrate material within the fluidized bed coating unit;
Initiating fluidizing air through the air inlet of the air handling system, passing through filters to prepare the substrate material for coating;
Heating, by the heating unit, the fluidizing air;
Loading the plasticizer material into the plasticizer container that is operatively connected to the peristaltic pump;
Loading the coating powder into the first chamber and the anti-adherent material into the second chamber of the coating material reservoir;
Activating the peristaltic pump and the air compressor to spray the plasticizer material onto the substrate material through the concentric air and plasticizer nozzle feeder; and
Operating the air compressor to regulate airflow through the air regulator, thereby transporting the coating powder from the first chamber or the anti-adherent material from the second chamber, as required, to the fluidized bed coating unit via the coating powder pneumatic feeder, where it is applied to the substrate material.
